(19) United States
(12) Patent Application Publication
(10) Pub. No.: US 2011/0144218 A1
(43) Pub. Date: Jun. 16, 2011

(54) Taste-Modified Consumable Products And Methods Of Preparation

(76) Inventors: David POSNER, Capitola, CA (US); Noah ABELSON, San Mateo, CA (US)

(21) Appl. No.: 12/965,869

(22) Filed: Dec. 11, 2010

Related U.S. Application Data
(60) Provisional application No. 61/285,898, filed on Dec. 11, 2009.

(51) Int. Cl. A23L 1/222; A23C 23/00; A23C 9/123; A23G 9/42; A23L 2/56; A23G 3/48; A61K 47/46

(52) U.S. Cl. 514/783; 426/650; 426/580; 426/583; 426/565; 426/615; 426/442

(57) ABSTRACT

A taste-modified consumable product, such as food product (including but not limited to fruit juice concentrates, fruit purées, and dairy products such as yogurt and ice cream), medical product, romance product, or entertainment product, comprising of a taste-modifying substance, such as extract from a miracle berry, and a method for production thereof.

### FIELD OF THE INVENTION

This invention relates to the use of taste-modifying substances, such as miracle berry extract, to improve the taste of consumable products. Consumable products include but are not limited to medicinal products, pharmaceutical products, diet products, romance products, entertainment products, recreational products, and food products (including but not limited to fruit juice concentrates, fruit purées , and dairy products such as yogurt and ice cream).

### BACKGROUND OF THE INVENTION

The botanical plant species of Synsepalum dulcificum is indigenous to tropical West Africa, where it is often referred to as “Sweet berry” or “Tàami berry” or Tahmè berry” and has been consumed for over 300 years. The fruit of this plant species has also been referred to as “miracle berry” or “miracle fruit.” The plant, which grows in the form of a shrub, yields ripe red berries consistently from December to June and inconsistently throughout the rest of the year—the berries being ellipsoidal in shape and about 0.75 inch long. The fruit has a palatable skin and thin layer of pulp surrounding a single large seed. It has been recognized for over 200 years that these berries have the unique property of modifying the taste of sour and bitter foods, drinks, and products to make such foods, drinks, and products taste sweet after the pulp has been chewed. It has been found that a component in the fruit depresses or masks the sour or bitter taste and accentuates the sweet taste of any normally sour or biter food eaten within a short period (approximately one hour) after first contacting the tongue with the pulp of fresh miracle fruit—resulting in a pleasantly sweet taste. By exposing the taste receptors on the tongue to the miracle berry, any sour or bitter tasting food, drink, or product can be made to taste sweet without the addition of sugar or artificial sweeteners. For example, fresh lemon can be made to taste pleasantly sweet by first eating a miracle berry. It is thought that the taste-modifying component in the miracle berry known as miraculin binds to the taste-receptors, thus altering the sensory perception of the sour and bitter taste in foods, drinks, and products eaten after the miracle fruit. There are additional natural sweeteners and taste modifiers that have been discovered but are not currently used commonly in food products today. These sweeteners and taste modifiers include but are not limited to Thaumatin, Monellin, Dioscoreophylim cumminisu (Serendipity Berry), Acer saccharum (sugar maple), Lippiz dulcis, Periandra dulcis, and Saccharum officinarum.

However, individuals that wish to enjoy the effects of the miracle berry and other natural sweeteners and taste modifiers with consumable products such as food products, medical products, diet products, romance products, entertainment products, and recreational products still need to chew the miracle berry or otherwise take the miracle berry extract before tasting the food product. Therefore, there remains a need in the art for the effective preparation of food products, medical products, diet products, romance products, entertainment products, and recreational products that contain the extract from the miracle berry (or other taste modifier) within the product itself.

### SUMMARY OF THE INVENTION

In one embodiment, the invention provides for taste-modified consumable products that comprise a taste-modifying component. For example, the consumable product can be a food product. In addition, the taste-modifying component can include a miracle berry-extract component. Moreover, the food product can be a fruit-based component, and the fruit-based component can be acidic. For example, the fruit-based component can include a fruit concentrate or a fruit puree. In addition, the acidic component can be or include citric acid, ascorbic acid, or acetic acid. The components of the taste-modified food product can be organic.

The invention also provides for taste-modified food products that include a dairy-based component. For example, the dairy-based component can include yogurt, Greek yogurt, or ice cream. The dairy-based component can also include a fruit-based component. The components of the taste-modified food product can be organic.

The invention also provides for taste-modified consumable products that are medicinal products. The invention includes medicinal products that include a miracle berry-extract component. The medicinal products of the invention can include, but are not limited to sprays, film strips, tablets, capsules, gels, soft candy, hardy candy, and powder.

The invention also provides for taste-modified consumable products that are diet products. The invention includes diet products that include a miracle berry-extract component. The diet products of the invention can include, but are not limited to, sprays, film strips, tablets, capsules, gels, soft candy, hardy candy, and powder.

The invention also provides for taste-modified consumable products that are romance products. The invention includes romance products that include a miracle berry-extract component. The romance products of the invention can include, but are not limited to, sprays, film strips, tablets, capsules, gels, soft candy, hardy candy, and powder.

The invention also provides for taste-modified consumable products that are entertainment products. The invention includes entertainment products that include a miracle berry-extract component. The entertainment products of the invention can include, but are not limited to, sprays, film strips, tablets, capsules, gels, soft candy, hardy candy, and powder.

In a second embodiment, the invention provides for methods of producing taste-modified consumable products by combining a consumable product with a miracle berry extract, or other taste-modifying component. The method includes consumable products that are food products. For example, the food product can be a fruit component, such as a fruit concentrate or a fruit puree. In addition, the method can include where the fruit-based component is an acidic component, such as citric acid, ascorbic acid, or acetic acid. In other aspects of these methods, the food component can be dairy based, such as with yogurt, Greek yogurt, or ice cream. In other aspects of the invention, the dairy-based food product also includes a fruit-based component, which may or may not be organic. In other aspects of the invention, the method produces taste-modified medicinal products. In yet other aspects of the invention, the method produces taste-modified diet products. In other aspects of the invention, the method produces taste-modified romance products. In addition, the invention provides for methods of producing taste-modified entertainment products.

### DETAILED DESCRIPTION OF THE INVENTION

As used herein, the singular forms “a,” “an,” and “the” include plural referents unless the context clearly dictates otherwise.

As used herein, the term “consumable product” encompasses any edible product, and is intended to have a broad meaning and encompasses formulated products intended to be consumed and ingested as well as products which are not normally intended to be, but can be, swallowed. Consumable products include, but are not limited to, food products, medicinal products, pharmaceutical product, diet products, romance products, entertainment products, and recreational products.

As used herein, the term “food product” encompasses any nourishing substance that is eaten, drunk, or otherwise taken into the body to sustain life, provide energy, promote growth, etc. However, a consumable product can fall within the definition of food product even if it is taken into the body for reasons other than nourishment. For example, food products encompass such materials as confections, icings, puddings and custards, baked goods, batters, frozen foods, sauces, sprays, gels, soft candy, hard candy, pills, liquids, lip balms, fresh fruits, frozen fruits and the like, which can be provided in final edible form after further cooking applications. Examples of food products encompassed by this invention include fruit concentrates, which can be consumed themselves or used to produce fruit juice and other fruit drinks and edible substances; fruit purees, which can be consumed themselves or used to produce other fruit drinks or edible substances; and dairy products, including yogurt and ice cream. Moreover, the food products encompassed by this invention include, but are not limited to, dairy products that are produced with fruit concentrates or fruit purees.

As used herein, the terms “fruit,” “fruit concentrate,” and “fruit puree” encompass both the traditional understanding of the term fruit, i.e., anything that comes from a flower (including both citrus and non-citrus fruits), but also is meant to encompasses other food that are traditionally or non-traditionally considered fruits, and even includes some vegetables. For example, “fruits” as encompassed by this application include, but are not limited to all varieties of apples, berries, melons, and citrus fruits. Therefore, fruits are meant to include, but are not limited to, all varieties of the following: apple, cranberry, pear, peach, plum, apricot, nectarine, grape, cherry, currant, raspberry, gooseberry, blackberry, blueberry, strawberry, lemon, lime, mandarin, orange, grapefruit, tomato, pineapple, custard-apple, coconut, pomegranate, guanabana, kiwi, mango, papaya, banana, watermelon, and cantaloupe, and feijoa (pineapple guava). Fruits are also meant to encompass some vegetables, such as potatoes, lettuce, celery, spinach, cabbage, broccoli, cauliflower and carrots.

As used herein “dairy products” refers to any product produced from milk or milk products, including but not limited to ice cream, yogurt, cheese, butter, sour cream, and the like. For the purposes of this application, dairy products also encompasses food products that are not produced from milk or milk products, but are non-dairy equivalents of food products that traditionally are dairy products, such as non-dairy ice cream and non-dairy yogurt.

As used herein, an “acidic” component can be any non-toxic acid, including naturally occurring carboxylic acids such as citric, malic, ascorbic, formic, acetic, tartaric, or inorganic acids or mixes thereof. It should be understood that the acids can be added as pure acids, salts, hydrates, solvates, etc. or in their natural form. Thus, the acids can be added to the food products as lime, lemon or apple juice, or the like.

As used herein, “medicinal product” encompasses any product that is consumed for medicinal purposes. The medicinal purpose can either be preventative, for treatment of a disease or ailment, or for stabilizing a disease or ailment to prevent spread or increased pain or harm. Medicinal products include pharmaceuticals as well as homeopathic drugs, and can take the form of pills, gels, gel caps, tablets, liquid medicine, syrups, spray, film strip, soft candy, hard candy, powder, and other pharmaceutical and homeopathic drugs. The medicinal product may or may not be foul tasting without the use of a taste-modifying component.

As used herein, “romance product” encompasses any product that is used in an intimate context either by individuals, with couples, or in groups. Romance products include, but are not limited to, lubricants and coated sex toys or other sexual aides.

As used herein, “entertainment product” encompasses any product that is consumed for entertainment purposes. For example, and entertainment product can be a game or experience, or part of a game or experience. In addition, an entertainment product can involve a recreational component, for example, a cocktail at a bar, club, or event. For example, the entertainment product can be, but is not limited to, a liquor product or an alcoholic drink.

As used herein, “taste-modifying component” refers to a composition that is able to modify the taste of a consumable product that is consumed concurrent or subsequent to the taste-modifying component. The taste-modifying components of the present invention can be derived from the plant species Synsepalum dulcificum, the fruit of which has been referred to as “miracle berry,” “miracle fruit,” “Sweet berry” or “Tàami berry” or “Tahmè berry.” The taste-modifying component can either be the miracle berry itself, the pulp thereof, or extracts of the miracle berry. The taste-modifying components also include, but are not limited to, thaumatin, monellin, Dioscoreophylim cumminisu (Serendipity Berry), Acer saccharum, Lippiz dulcis, Periandra dulcis, and Saccharum officinarum. Other taste-modifying components will be understood to be encompassed by this invention.

While the berries of the Synsepalum species can be used to create a pulp, an extract of that pulp can also be easily created and are contemplated in the methods and compositions of this invention. As used herein, the term “miracle berry extract” means any product of a method to purify, partially purify, modify, stabilize, infuse, or combine the component of the fruit that induces a sweet sensation while consuming acidic and bitter flavors after coating the tongue. This component is thought to be a glycoprotein known as Miraculin, but the invention is not to be limited by this understanding. Miracle-berry extracts also encompass the chemical synthesis of the protein miraculin, the production of the material by recombinant methods, and a heat stabilized form of the protein. The use of pulp can be substituted for extract and vice versa. The extract can be in many types of forms, including but not limited to powder, liquid, spray, pill, gel, hard candy, soft candy, frozen fruit, dried fruit, liquids, and lip balm. For example, U.S. Pat. No. 3,824,323 to Harvey et al., U.S. Pat. No. 3,995,031 to Henkin et al., and U.S. Pat. No. 5,886,155 to Armah et al. set forth numerous methods of purification and are incorporated herein by reference in their entirety. The miracle berry extract can also be in the form of a lyophilized powder. Additional methods of extraction, isolation, and or preparation will be understood and within the level of skill in the relevant arts and are intended to be encompassed by the present invention.

The food products and methods of this invention can be used in situations where the presence or addition of sweeteners is not desired. For example, the food products of this invention will be useful for diabetics, individuals that cannot tolerate natural or synthetic sweeteners, and individuals that are trying to avoid natural or synthetic sweeteners. However, the food products and methods of this invention are not limited to these uses, and can include food products or methods that naturally contain sweeteners, or have sweeteners added to them. The products and methods can also include a gel to be added to a medicine, an extract to be used to coat a pill, a spray to be used/applied to the tongue before a meal, a concentrate to be added to a soft candy, a liquid to coat a hard candy, a lip balm used day to day, a frozen or dried fruit to be added to a cocktail or a powder to be rimmed on a glass, a concentrate to be combined with a lubricant or applied to a sex toy, a chewable tablet to consume before a meal, or a candy form to be incorporated into a game. Moreover, this invention can be practiced with components that are all organic in order to provide a food product that can be certified organic.

### EXAMPLE 1

### Blueberries

A puree of blueberries was prepared from a starting amount of 83.7 grams. The amount of resulting pureed fruit was 59.4 grams. 15.2 gram of lemon juice and 1 gram lyophilized miracle berry powder was added to the pureed fruit. The resulting amount of taste-modified pureed fruit was approximately 70 grams.

A test subject placed a spoonful of the taste-modified pureed fruit in their mouth immediately after preparation and observed that the preparation was “tart.” After 40 seconds, the test subject repeated the tasting and observed that the taste-modified pureed fruit was “not as tart.” After an additional 45 seconds, the test subject repeated the tasting and observed that the taste-modified pureed fruit was “good.” After an additional 30 seconds, the test subject tasted a fourth spoonful of taste-modified pureed fruit and observed that it was “sweet.” After an additional 30 seconds, the test subject tasted a fifth spoonful of taste-modified pureed fruit and observed that it was “sweet, sweet” and that the “aftertaste was sweeter.” Finally, after an additional 40 seconds, the test subject tasted a sixth spoonful of taste-modified pureed fruit and observed that it was “sweet, sweet, sweet.”

### EXAMPLE 2

### Strawberries

A puree of strawberries was prepared from a starting amount of 89 grams. The amount of resulting pureed fruit was 63 grams. 13.2 gram of lemon juice and 1 gram lyophilized miracle berry powder was added to the pureed fruit. The resulting amount of taste-modified pureed fruit was approximately 73 grams.

A test subject placed a spoonful of the taste-modified pureed fruit in their mouth immediately after preparation and observed that the preparation was “sour and slightly sweet.” After 35 seconds, the test subject repeated the tasting and observed that the taste-modified pureed fruit was “slightly sweet, but could “sense the flavor change of the miracle berry.” After an additional 30 seconds, the test subject repeated the tasting and observed that the taste-modified pureed fruit was “sweet.” After an additional 30 seconds, the test subject tasted a fourth spoonful of taste-modified pureed fruit and observed that it was still “sweet.” Finally, after an additional 30 seconds, the test subject tasted a fifth spoonful of taste-modified pureed fruit and observed that it was “sweet, sweet, but still maintained its flavor and tang.”

### EXAMPLE 3

### Mangos

A puree of mangos was prepared from an unrecorded starting amount. The amount of resulting pureed fruit was 72 grams. 14 grams of lemon juice and 1 gram lyophilized miracle berry powder was added to the pureed fruit. The resulting amount of taste-modified pureed fruit was approximately 76 grams.

A test subject placed a spoonful of the taste-modified pureed fruit in their mouth immediately after preparation and observed that the preparation was “pretty good, slightly sweet, and tart.” After 30 seconds, the test subject repeated the tasting and observed that the taste-modified pureed fruit was “sweeter than the previous taste.” After an additional 30 seconds, the test subject repeated the tasting and observed that the taste-modified pureed fruit was “still sweeter.” After an additional 30 seconds, the test subject tasted a fourth spoonful of taste-modified pureed fruit and observed that it was “sweet, and not tart at all.” After an additional 10 seconds, the test subject tasted a fifth spoonful of taste-modified pureed fruit and observed that it was “sweet.” Finally, after an additional 25 seconds, the test subject tasted a sixth spoonful of taste-modified pureed fruit and observed that it was “as sweet as anyone would want.”

### EXAMPLE 4

### Green Apple

A puree of green apples was prepared from an unrecorded starting amount. The amount of resulting pureed fruit was 53 grams. 15.3 gram of lemon juice and 1 gram lyophilized miracle berry powder was added to the pureed fruit. The resulting amount of taste-modified pureed fruit was approximately 69 grams.

A test subject placed a spoon full of the taste-modified pureed fruit in their mouth immediately after preparation and observed that the preparation was “incredibly sour.” After 30 seconds, the test subject repeated the tasting and observed that the taste-modified pureed fruit was “better tasting.” After an additional 35 seconds, the test subject repeated the tasting and observed that the taste-modified pureed fruit was still “better, with a tart taste still present.” After an additional 60 seconds, the test subject tasted a fourth spoonful of taste-modified pureed fruit and observed a taste of “sweet lemon juice and apples, (the apples taste the same.” Finally, after an additional 70 seconds, the test subject tasted a fifth spoonful of taste-modified pureed fruit and observed that the flavor was “sweet.”

### EXAMPLE 5

### Blackberries

A puree of blackberries was prepared from an unrecorded starting amount. The amount of resulting pureed fruit was 88 grams. 16.3 gram of lemon juice and 1.05 gram lyophilized miracle berry powder was added to the pureed fruit. The resulting amount of taste-modified pureed fruit was approximately 103 grams.

A test subject placed a spoonful of the taste-modified pureed fruit in their mouth immediately after preparation and observed that the preparation was “tart and very sour.” After 30 seconds, the test subject repeated the tasting and observed that the taste-modified pureed fruit was “better, but still tart.” After an additional 30 seconds, the test subject repeated the tasting and observed that the taste-modified pureed fruit was “sweet, but still a little tart.”After an additional 60 seconds, the test subject tasted a fourth spoonful of taste-modified pureed fruit and observed that it was “sweet.” Finally, after an additional 70 seconds, the test subject tasted a fifth spoonful of taste-modified pureed fruit and observed that it was “overly sweet.”

### EXAMPLE 6

### Crushed Tomato

A puree of crushed tomato was prepared from an unrecorded starting amount. The amount of resulting pureed fruit was 85 grams. Lemon juice and 1 gram lyophilized miracle berry powder was added to the pureed fruit. The result was a taste-modified pureed fruit food product.

A test subject placed a spoonful of the taste-modified pureed fruit in their mouth immediately after preparation and observed that the preparation was “fresh but not sweet, and acidic.” After 30 seconds, the test subject repeated the tasting and observed that the taste-modified pureed fruit was “slightly sweet.” After an additional 30 seconds, the test subject repeated the tasting and observed that the taste-modified pureed fruit was “sweeter.” After an additional 30 seconds, the test subject tasted a fourth spoonful of taste-modified pureed fruit and observed that it was “a sweet fresh sauce.” After an additional 30 seconds, the test subject tasted a fifth spoonful of taste-modified pureed fruit and observed that it was “fresh and sweet.” Finally, after an additional 30 seconds, the test subject tasted a sixth spoonful of taste-modified pureed fruit and observed that it was “too sweet for a sauce.”

### EXAMPLE 7

### Cooked And Chopped Tomatoes With Salt

A puree of cooked and chopped tomatoes was prepared with salt. Lemon juice and 2 grams of lyophilized miracle berry powder was added to the pureed fruit. The resulting amount of taste-modified pureed fruit was approximately 150 grams.

A test subject placed a spoonful of the taste-modified pureed fruit in their mouth immediately after preparation and observed that the preparation was “a nice tomato sauce and not sweet.” After 30 seconds, the test subject repeated the tasting and observed that the taste-modified pureed fruit was “sweeter.” Finally, after an additional 30 seconds, the test subject repeated the tasting and observed that the taste-modified pureed fruit was “too sweet.”

### EXAMPLE 8

### Pineapple

A puree of pineapple was prepared from an unrecorded starting amount. The amount of resulting pureed fruit was 88 grams. 17.6 gram of lemon juice and 1.26 gram lyophilized miracle berry powder was added to the pureed fruit. The result was a taste-modified pureed fruit food product.

A second puree of pineapple was prepared from an unrecorded starting amount. This pineapple was less sweet than the previous one. The amount of resulting pureed fruit was 81 grams. Only 9 gram of lemon juice was added because it was already acidic. 1 gram lyophilized miracle berry powder was added to the pureed fruit. The result was also a taste-modified pureed fruit food product.

### EXAMPLE 9

### Raspberries

A puree of raspberries was prepared from an unrecorded starting amount. The amount of resulting pureed fruit was 80 grams. 15.3 gram of lemon juice and 0.99 gram lyophilized miracle berry powder was added to the pureed fruit. The resulting amount of taste-modified pureed fruit was approximately 95 grams.

### EXAMPLE 10

### Greek Yogurt

A sample of 1 commercial cup of Greek yogurt was used. 1 gram lyophilized miracle berry powder was added to the yogurt. The result was a taste-modified yogurt food product.

A second sample of 88 grams of Greek yogurt was prepared. 1 gram lyophilized miracle berry powder was added to the yogurt. The result was a taste-modified yogurt food product.

A test subject placed a spoonful of this second taste-modified yogurt in their mouth immediately after preparation and observed that the preparation was “tart.” After 30 seconds, the test subject repeated the tasting and observed that the taste-modified yogurt was “sweet but still tart.” After an additional 30 seconds, the test subject repeated the tasting and observed that the taste-modified yogurt tasted “like cream” and was “pleasantly sweet.” Finally, after an additional 30 seconds, the test subject tasted a fourth spoonful of taste-modified yogurt and observed that it was “sweet enough to be a stand-alone dessert.”

### EXAMPLE 11

### Boiled Golden Delicious Apple

A puree of boiled golden delicious apple was prepared from an unrecorded starting amount. The amount of resulting pureed fruit was 150 grams. 25 grams of lemon juice and 1.5 gram lyophilized miracle berry powder was added to the pureed fruit. The result was a taste-modified pureed fruit food product.

A test subject placed a spoonful of the taste-modified pureed fruit in their mouth immediately after preparation and observed that the preparation was “tart.” After 30 seconds, the test subject repeated the tasting and observed that the taste-modified pureed fruit was “sweet and tart.” After an additional 30 seconds, the test subject repeated the tasting and observed that the taste-modified pureed fruit was “awesomely sweet.” Finally, after an additional 30 seconds, the test subject tasted a fourth spoonful of taste-modified pureed fruit and observed that it was “like apple pie filling.”

### EXAMPLE 12

### Kiwi

A puree of kiwi was prepared from an unrecorded starting amount. The amount of resulting pureed fruit was 83 grams. 15.6 gram of lemon juice and 1 gram lyophilized miracle berry powder was added to the pureed fruit. The result was a taste-modified pureed fruit food product.

### EXAMPLE 13

### Ice Cream

A sample of ice cream was used. Miracle berry powder was added to the ice cream. The result was a taste-modified ice-cream food product.

### Example 14

### Medicinal Gel

Miracle berry extract is added to a gel. A room temperature stable gel containing the miracle berry extract is added to a liquid medicine. The gel is added either before bottling the liquid medicine by the manufacturer or is combined by the consumer once in possession of the liquid. The miracle berry gel is mixed by stirring or some other way. Combining the miracle fruit gel with the medicine sweetens and improved the taste of the medicine. The miracle berry gel can also be consumed before the user consumes the medicinal liquid so that the miracle berry gel makes contact with the tongue prior to medicine consumption.

### Example 15

### Medicinal Coating

Miracle fruit extract is applied in either liquid or powder form as a coating on a pill. The coating is either prepared by the manufacturer by coating the pill with a liquid extract of the miracle berry, or the end user applies the miracle fruit extract powder to the pill. The result is an improved taste of the pill without using sugar or a sweetener, because the miracle berry extract makes contact with the users tongue and improves the taste, thereby masking the foul taste of the pill.

### Example 16

### Pre-Meal Spray

Miracle berry extract is added to a liquid. The liquid form of the miracle berry extract is sprayed onto the tongue before consuming a snack, drink, or meal. The user sprays the liquid form of the extract onto his or her tongue while preparing food, or purchasing a snack, or a minute or two before consuming a beverage. 30 seconds following the spraying of the liquid form of the extract, the user encounters an improved and sweetened taste of whatever it is he or she is consuming. Alternatively, the liquid form of the extract can be sprayed directly onto the snack, drink, or meal.

### Example 17

### Soft Candy

Miracle Berry extract is incorporated into a soft candy. The extract can be in concentrate form. The miracle berry extract is either incorporated by the manufacturer into the soft candy mixture or ingredients, or it is included as a separate “miracle-berry chamber” within the candy. The miracle berry extract will make direct contact with the tongue once the consumer either bites into the candy or opens the miracle-berry chamber. The candy is consumed before a meal, in conjunction with candy or fruit.

### Example 18

### Liquid Candy Coating

Miracle berry extract is added to a liquid. The liquid form of the miracle berry extract is applied as a coating on a hard candy or lollipop. The miracle berry extract is either incorporated into the hard candy or lollipop and packaged by the manufacturer or the end user can apply the liquid coating to the hard candy or lollipop. The user's tongue will make contact with the coating prior to making contact with the hard candy or lollipop thereby improving the taste of the lollipop and any subsequently consumed products.

### Example 19

### Lip Balm

Miracle Berry extract is combined into a lip balm recipe. The finished product is applied to the users lips under normal conditions for a lip balm, but the user has improved taste of subsequent food products consumed. The miracle berry lip balm makes contact with the tongue thereby modifying the taste perception of the user.

### Example 20

### Cocktails

A frozen or dried piece of the miracle fruit is served as a garnish on a cocktail. The powder form of the miracle fruit is used to rim a shot or cocktail glass. In the case of the dried or frozen piece of fruit, the fruit is consumed either before the drink or after the drink has been partially consumed in order to experience a taste modification mid-drink. The frozen or dried fruit is sucked before spitting out the seed. In the case of the powder, the glass is rimmed on and the powder is consumed as the glass is brought into contact with the tongue/mouth. In both cases, the miracle berry is used to modify and improve the taste of a cocktail—preferably one that contains a limited amount of sugar or other sweeteners. The miracle berry extract can also be added directly to the cocktail to improved the taste without additional sugar or sweeteners.

### Example 21

### Lubricant Or Sex Toy

Miracle berry extract is combined with the ingredients of a lubricant or applied to a sex toy. When incorporated into a lubricant, the lubricant can be consumed. The miracle berry extract-containing lubricant makes contact with the consumers tongue and transforms the taste of their romantic partner. For example, in the case of kissing, their partners kiss will taste sweeter. In the case of oral sex, their romantic partner's genitalia and related fluids will taste sweeter. The coating on the sex toy provides the same benefit and effect, but the user licks or sucks on the toy before using it on his or her partner.

### EXAMPLE 22

### Chewable Tablet Before A Meal

Miracle berry extract is combined with a chewable tablet. This chewable tablet form of the Miracle Berry extract is chewed while preparing or purchasing a meal. The chewing and/or sucking will coat the tongue of the consumer. Once the tablet has been completely dissolved, the consumer eats a meal, snack, or drinks a beverage whose taste will improve in due to the taste-modification capability of the Miracle Berry.

### EXAMPLE 23

### Candy During A Game

A miracle berry candy is consumed before, during, or after a game and/or experience. The taste modification effect is featured in a game or experience because of its novelty or “trippy” appeal. The taste modification effect can be used to but need not be limited to tricking or surprising a player or participant in the game and/or experience. The Miracle Berry candy can be either a hard or soft candy with a Miracle Berry filling, coating, or ingredient in the recipe. This is preferentially a blindfolded taste tests.

All patents, patent applications, and other scientific or technical writings referred to anywhere herein are incorporated by reference in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

What is claimed is:

1. A taste-modified consumable product, wherein the consumable product comprises a taste-modifying component.

2. The taste-modified consumable product of claim 1, wherein the consumable product is a food product.

3. The taste-modified consumable product of claim 2, wherein the taste-modifying component is a miracle berry-extract.

4. The taste-modified consumable product of claim 3, wherein the food product comprises a fruit-based component.

5. The taste-modified consumable product of claim 4, wherein the fruit-based component is selected from the group consisting of fruit concentrate and fruit puree.

6. The taste-modified consumable product of claim 4, wherein the fruit-based component comprises an acidic component.

7. The taste-modified consumable product of claim 6, wherein the acidic component is selected from the group consisting of citric acid, ascorbic acid, and acidic acid.

8. The taste-modified consumable product of claim 4, wherein the fruit-based component is organic.

9. The taste-modified consumable product of claim 3, wherein the food product comprises a dairy-based component.

10. The taste-modified consumable product of claim 9, wherein the dairy-based component is selected from the group consisting of yogurt, Greek yogurt, and ice cream.

11. The taste-modified consumable product of claim 10, further comprising a fruit-based component.

12. The taste-modified consumable product of claim 11, wherein the fruit-based component is organic.

13. The taste-modified consumable product of claim 1, wherein the consumable product is a medicinal product.

14. The taste-modified consumable product of claim 13, wherein the taste-modifying component is a miracle berry extract.

15. The taste-modified consumable product of claim 14, wherein the form of the medicinal product is selected from the group consisting of spray, film strip, tablet, capsule, gel, soft candy, hard candy, and powder.

16. The taste-modified consumable product of claim 1, wherein the consumable product is a romance product.

17. The taste-modified consumable product of claim 16, wherein the taste-modifying component is a miracle berry extract.

18. The taste-modified consumable product of claim 17, wherein the form of the romance product is selected from the group consisting of spray, film strip, tablet, capsule, gel, soft candy, hard candy, and powder.

19. The taste-modified consumable product of claim 1, wherein the consumable product is an entertainment product.

20. The taste-modified consumable product of claim 19, wherein the taste-modifying component is a miracle berry extract.

21. The taste-modified consumable product of claim 20, wherein the form of the entertainment product is selected from the group consisting of spray, film strip, tablet, capsule, gel, soft candy, hard candy, and powder.

22. A method of producing a taste-modified consumable product comprising combining a consumable product with a miracle berry extract.

23. The method of claim 22, wherein the consumable product is a food product.

24. The method of claim 23, wherein the food product comprises a fruit-based component.

25. The method of claim 24, wherein the fruit-based component is selected from the group consisting of fruit concentrate and fruit puree.

26. The method of claim 25, wherein the fruit-based component comprises an acidic component.

27. The method of claim 26, wherein the acidic component is selected from the group consisting of citric acid, ascorbic acid, and acidic acid.

28. The method of claim 23, wherein the food product comprises a dairy-based component.

29. The method of claim 28, wherein the dairy-based component is selected from the group consisting of yogurt, Greek yogurt, and ice cream.

30. The method of claim 28, wherein the food product further comprises a fruit-based component.

31. The method of claim 30, wherein the fruit-based component is organic.

32. The method of claim 23, wherein the consumable product is a medicinal product.

33. The method of claim 23, wherein the consumable product is a romance product.

34. The method of claim 23, wherein the consumable product is an entertainment product.
